(19) United States
(12) Patent Application Publication
(10) Pub. No.: US 2023/0054959 A1
(43) Pub. Date: Feb. 23, 2023

(54) Nasal Device

(71) Applicant: Nina D. Farzin, Potomac, MD (US)

(72) Inventor: Nina D. Farzin, Potomac, MD (US)

(21) Appl. No.: 17/894,091

(22) Filed: Aug. 23, 2022

Related U.S. Application Data
(60) Provisional application No. 63/236,150, filed on Aug. 23, 2021.

(51) Int. Cl. A61M 3/02; A61M 1/00

(52) U.S. Cl. CPC A61M 3/0279; A61M 1/682; A61M 3/0262; A61M 1/631; A61M 1/80; A61M 2210/0618; A61M 2205/076; A61M 2205/7545; A61M 2205/7509; A61M 2205/7518; A61M 2206/10; A61M 2205/59

(57) ABSTRACT

A nasal device includes a tip member having a first end and a second end, the second end of the tip member being oppositely disposed relative to the first end, the tip member including a first wall disposed at the first end thereof, the first wall defining a first aperture configured to be in fluid communication with a nostril of a person; a cover member disposed over a portion of the tip member, the cover member including a second wall overlapping at least a portion of the first wall of the tip member, the second wall defining a second aperture configured to be in fluid communication with the nostril of the person and the first aperture of the tip member, the cover member configured to provide a seal with the nostril of the person; and a vacuum source or a fluid container coupled to the tip member.

### CROSS-REFERENCE TO RELATED APPLICATIONS

This patent application claims priority to, and incorporates by reference in its entirety, U.S. Provisional Patent Application No. 63/236,150, entitled “Nasal Aspirator”, filed on Aug. 23, 2021.

### STATEMENT REGARDING FEDERALLY SPONSORED RESEARCH OR DEVELOPMENT

Not Applicable.

### NAMES OF THE PARTIES TO A JOINT RESEARCH AGREEMENT

Not Applicable.

### INCORPORATION BY REFERENCE OF MATERIAL SUBMITTED ON A COMPACT DISK

Not Applicable.

### BACKGROUND OF DISCLOSURE

### 1. Field of the Disclosure

The disclosure generally relates to a nasal device for removing mucus from a nostril or irrigating a nasal passage. More particularly, the disclosure relates to a nasal aspirator that uses a suction force to remove mucus from a nostril, and a nasal irrigation device that comprises a fluid container for holding a liquid solution used to irrigate a nasal passage.

### 2. Background

Newborns are delicate and their immune systems continue to develop as they grow. They are more susceptible to congestion, and the lack of ability to clear their nasal passages themselves leaves them especially prone to discomfort. A nasal aspirator is a device used to suction mucus from a baby's nasal passages. A baby's nasal passages are delicate; therefore, gentle suction and not going too deep are important. Failing to be cautious can result in nosebleeds, swelling, and additional discomfort. Conventional suction aspirators are hard to both assemble, as well as disassemble and clean. Some of these conventional suction aspirators use the mouth suction method to clear out nasal passages, which increases the risk of cross-contamination and germ transfer. Other conventional aspirators are overly complex, require a great deal of coordination on the part of the user, and cannot be easily used with just one hand. In addition, the overall effectiveness of these conventional nasal aspirators is limited because they do not form a good seal with the nostril of the person. Nasal irrigation devices have similar limitations and drawbacks.

Therefore, what is needed is a nasal device that is easy to disassemble and clean. Moreover, a nasal device is needed that includes a stop portion for preventing the device from being over-inserted into the nostril of the person. Furthermore, there is a need for a nasal device that forms an effective suction seal with the nostril of the person.

### SUMMARY OF THE DISCLOSURE

Accordingly, the present disclosure is directed to a nasal device that substantially obviates one or more problems resulting from the limitations and deficiencies of the related art.

In accordance with one or more embodiments of the present disclosure, there is provided a nasal device that includes a tip member having a first end and a second end, the second end of the tip member being oppositely disposed relative to the first end, the tip member including a first wall disposed at the first end thereof, the first wall defining a first aperture configured to be in fluid communication with a nostril of a person; a cover member disposed over a portion of the tip member, the cover member including a second wall overlapping at least a portion of the first wall of the tip member, the second wall defining a second aperture configured to be in fluid communication with the nostril of the person and the first aperture of the tip member, the cover member configured to provide a seal with the nostril of the person; and a vacuum source or a fluid container coupled to the tip member, the vacuum source configured to create a vacuum force through the first and second apertures so as to remove mucus from the nostril of the person, or the fluid container configured to store a liquid solution that is to be expelled through the first and second apertures and into the nostril of the person.

In a further embodiment of the present disclosure, the tip member further comprises a tapered distal end portion at the first end thereof, the tapered distal end portion of the tip member including the first wall and being disposed inside the cover member.

In yet a further embodiment, the tip member further comprises a stem portion connected to the tapered distal end portion.

In still a further embodiment, the stem portion of the tip member further comprises an annular protrusion disposed thereon, and the cover member further comprises a central passageway with an annular groove disposed in the central passageway, the annular protrusion on the stem portion of the tip member matingly engaging with the annular groove in the central passageway of the cover member so as to securely attach the cover member to the tip member.

In yet a further embodiment, the tip member further comprises an attachment collar portion at the second end thereof, the stem portion being connected between the tapered distal end portion of the tip member and the attachment collar portion of the tip member, and the attachment collar portion attaching the tip member to the vacuum source or the fluid container.

In still a further embodiment, the nasal device comprises the vacuum source, the vacuum source being in a form of an aspirator bulb member, the aspirator bulb member being attached to the attachment collar portion of the tip member, the aspirator bulb member configured to create the vacuum force for removing mucus from the nostril of the person and through the first and second apertures, the vacuum force being created as a result of the aspirator bulb member being initially compressed by a user, and then the user gradually releasing the compression of the aspirator bulb member.

In yet a further embodiment, the attachment collar portion of the tip member comprises one or more annular protrusions disposed thereon, the attachment collar portion of the tip member is received within an opening of the aspirator bulb member so as to attach the tip member to the aspirator bulb member, and the opening of the aspirator bulb member is circumscribed by a bounding wall with one or more annular grooves formed therein, the one or more annular protrusions on the attachment collar portion of the tip member matingly engaging with the one or more annular grooves in the bounding wall of the aspirator bulb member so as to securely attach the tip member to the aspirator bulb member.

In still a further embodiment, the nasal device comprises the vacuum source, the vacuum source being in a form of a mouth suction aspirator, and the mouth suction aspirator comprises a connector member that is attached to the attachment collar portion of the tip member; a filter member disposed in the connector member; and a mouthpiece connected to the connector member by means of a flexible tube, the mouth suction aspirator configured to create the vacuum force for removing mucus from the nostril of the person and through the first and second apertures when a user places the mouthpiece into his or her mouth and draws air through a distal end of the mouthpiece, the filter member configured to trap mucus from the nostril of the person so as to prevent the mucus, viruses, and/or bacteria from entering the mouth of the user.

In yet a further embodiment, the filter member of the mouth suction aspirator comprises a serpentine air path for trapping the mucus from the nostril of the person inside the filter member.

In still a further embodiment, the nasal device comprises the fluid container, the fluid container being in a form of a nasal irrigation bottle, the attachment collar portion of the tip member being part of a bottle cap member, the nasal irrigation bottle configured to be attached to the bottle cap member, and the nasal irrigation bottle configured to hold the liquid solution for being expelled through the first and second apertures and into the nostril of the person.

In yet a further embodiment, the nasal device further comprises a straw member extending from the tip member to a bottom of the nasal irrigation bottle so as to extract the liquid solution from the bottom of the nasal irrigation bottle.

In still a further embodiment, the nasal device comprises the fluid container, the fluid container being in a form of a nasal irrigation syringe, and the nasal irrigation syringe comprises a syringe body, the syringe body comprising a fluid reservoir for holding the liquid solution, and the tip member forming a part of the syringe body; and a plunger assembly slidably received within the syringe body, the plunger assembly configured to be slidably displaced in a longitudinal direction of the syringe body, and the plunger assembly configured to discharge the liquid solution from the nasal irrigation syringe and into the nostril of the person when a force is applied to an end of the plunger assembly by a user.

In yet a further embodiment, the plunger assembly comprises a plunger head portion, a plunger shaft having a cross-sectional profile, and a plunger handle portion, and the plunger handle portion of the plunger assembly is configured to receive the force applied by the user to discharge the liquid solution from the nasal irrigation syringe.

In still a further embodiment, the cover member further comprises a hemispherical protruding portion that includes the second wall, the hemispherical protruding portion operating as a primary stop portion sized to limit the depth that the second wall is able to be inserted into the nostril of the person, the hemispherical protruding portion further operating as a barrier between the tip member and the nostril of the person so as to enhance comfort for the person and prevent the tip member from directly contacting the nostril of the person, and a portion of the hemispherical protruding portion of the cover member configured to provide the seal with the nostril of the person.

In yet a further embodiment, the cover member further comprises a cover body portion connected to the hemispherical protruding portion, the cover body portion operating as a stabilizer when the hemispherical protruding portion is being inserted into the nostril of the person, and the cover body portion further operating as a secondary stop portion sized to prevent over-insertion of the nasal device into the nostril of the person.

In still a further embodiment, the cover body portion is in a shape of an animal head.

In yet a further embodiment, the animal head forming the cover body portion is a bear head.

In still a further embodiment, the tip member is formed from a first polymeric material, and the cover member is formed from a second polymeric material, the second polymeric material being more flexible than the first polymeric material.

In yet a further embodiment, the first polymeric material forming the tip member comprises polypropylene, and the second polymeric material forming the cover member comprises silicone.

In still a further embodiment, the tip member, the cover member, and the vacuum source or the fluid container are capable of being dissembled from one another for easy cleaning of the nasal device.

It is to be understood that the foregoing general description and the following detailed description are merely exemplary and explanatory in nature. As such, the foregoing general description and the following detailed description should not be construed to limit the scope of the appended claims in any sense.

### BRIEF DESCRIPTION OF THE SEVERAL VIEWS OF THE DRAWINGS

The device will now be described, by way of example, with reference to the accompanying drawings, in which:

FIG. 1 is a perspective view of a nasal device, according to a first illustrative embodiment of the disclosure, wherein the nasal device is in a form of a bulb-type nasal aspirator;

FIG. 2 is a front elevational view of the nasal device shown in FIG. 1;

FIG. 3 is a rear elevational view of the nasal device shown in FIG. 1;

FIG. 4 is a first side view of the nasal device shown in FIG. 1;

FIG. 5 is an opposite second side view of the nasal device shown in FIG. 1;

FIG. 6 is a top plan view of the nasal device shown in FIG. 1;

FIG. 7 is a bottom plan view of the nasal device shown in FIG. 1;

FIG. 8 is an exploded perspective view of the nasal device shown in FIG. 1;

FIG. 9 is a longitudinal sectional view of the nasal device shown in FIG. 1;

FIG. 10 is a perspective view of another nasal device, according to a second illustrative embodiment of the disclosure, wherein the nasal device is in a form of a mouth suction nasal aspirator;

FIG. 11 is a front elevational view of the nasal device shown in FIG. 10;

FIG. 12 is a rear elevational view of the nasal device shown in FIG. 10;

FIG. 13 is a first side view of the nasal device shown in FIG. 10;

FIG. 14 is an opposite second side view of the nasal device shown in FIG. 10;

FIG. 15 is a top plan view of the nasal device shown in FIG. 10;

FIG. 16 is a bottom plan view of the nasal device shown in FIG. 10;

FIG. 17 is an exploded perspective view of the nasal device shown in FIG. 10;

FIG. 18 is a perspective view of a filter member of the nasal device shown in FIG. 10;

FIG. 19 is a longitudinal sectional view of the filter member shown in FIG. 18;

FIG. 20 is a longitudinal sectional view of the nasal device shown in FIG. 10;

FIG. 21 is a perspective view of a yet another nasal device, according to a third illustrative embodiment of the disclosure, wherein the nasal device is in a form of a nasal irrigation bottle;

FIG. 22 is a front elevational view of the nasal device shown in FIG. 21;

FIG. 23 is a rear elevational view of the nasal device shown in FIG. 21;

FIG. 24 is a first side view of the nasal device shown in FIG. 21;

FIG. 25 is an opposite second side view of the nasal device shown in FIG. 21;

FIG. 26 is a top plan view of the nasal device shown in FIG. 21;

FIG. 27 is a bottom plan view of the nasal device shown in FIG. 21;

FIG. 28 is an exploded perspective view of the nasal device shown in FIG. 21;

FIG. 29 is a longitudinal sectional view of the nasal device shown in FIG. 21;

FIG. 30 is a perspective view of a still another nasal device, according to a fourth illustrative embodiment of the disclosure, wherein the nasal device is in a form of a nasal irrigation syringe;

FIG. 31 is a front elevational view of the nasal device shown in FIG. 30;

FIG. 32 is a rear elevational view of the nasal device shown in FIG. 30;

FIG. 33 is a first side view of the nasal device shown in FIG. 30;

FIG. 34 is an opposite second side view of the nasal device shown in FIG. 30;

FIG. 35 is a top plan view of the nasal device shown in FIG. 30;

FIG. 36 is a bottom plan view of the nasal device shown in FIG. 30;

FIG. 37 is a partially exploded perspective view of the nasal device shown in FIG. 30; and

FIG. 38 is a longitudinal sectional view of the nasal device shown in FIG. 30.

Throughout the figures, the same parts are always denoted using the same reference characters so that, as a general rule, they will only be described once.

### DETAILED DESCRIPTION OF ILLUSTRATED EMBODIMENT(S)

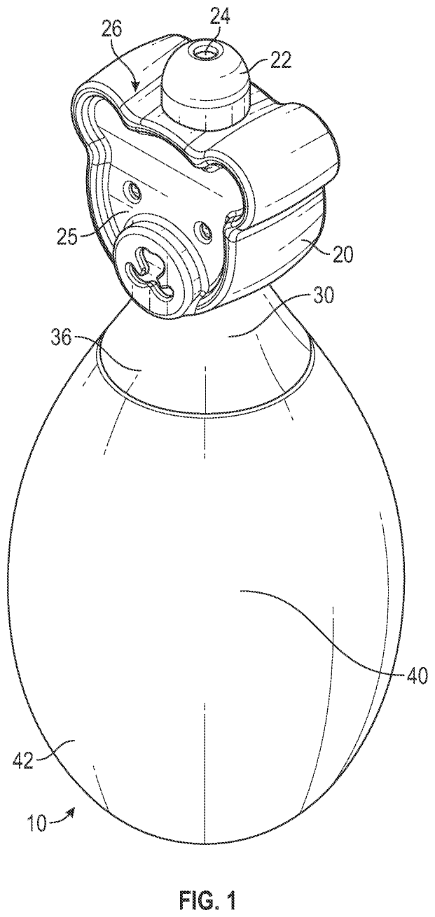

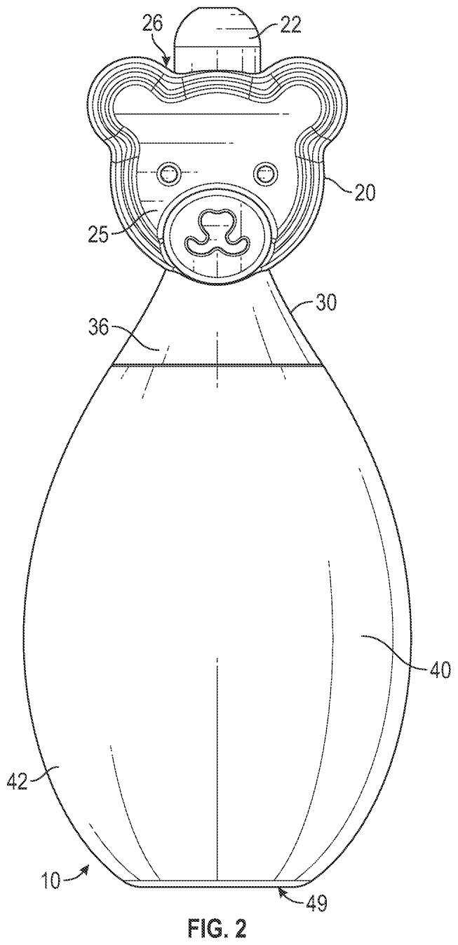

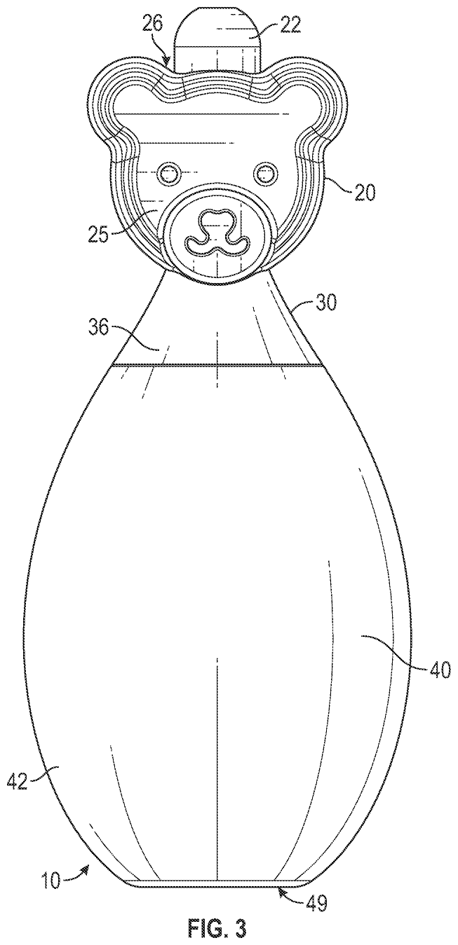

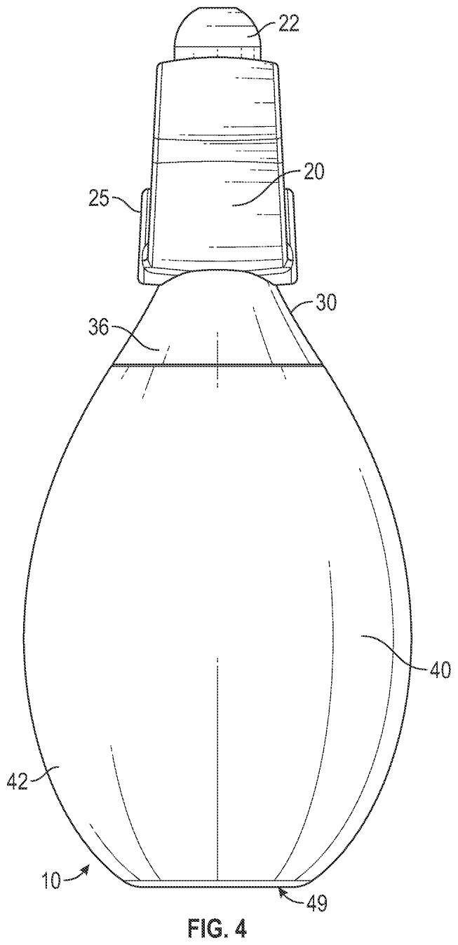

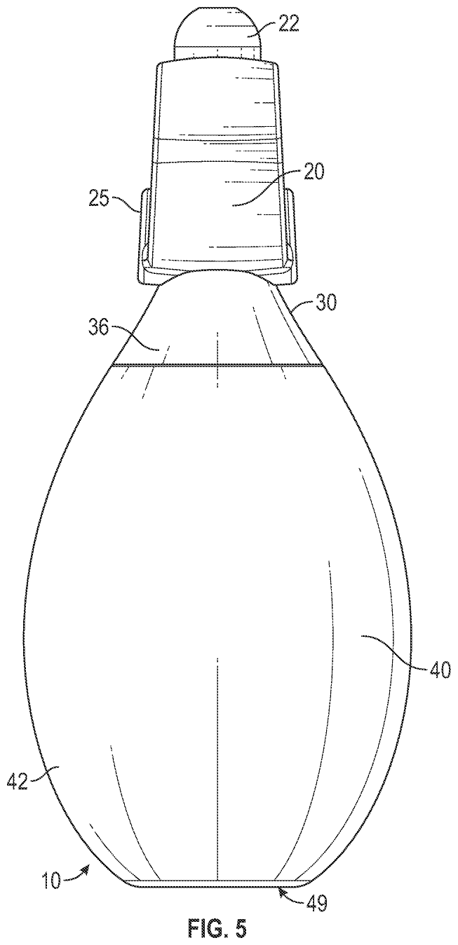

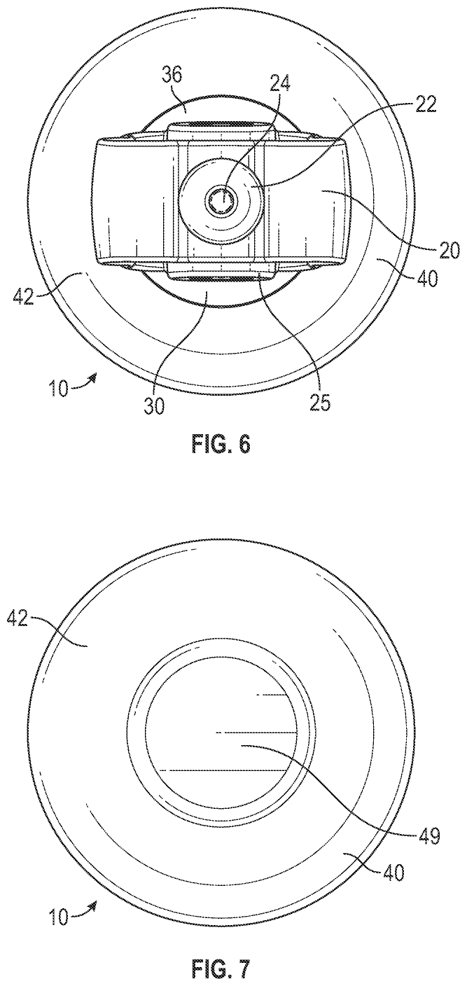

A first illustrative embodiment of a nasal device is seen generally at 10 in FIGS. 1-9. In the first illustrative embodiment, the nasal device is in a form of a bulb-type aspirator 10. With initial reference to FIGS. 1, 8, and 9, it can be seen that the nasal aspirator 10 generally comprises a tip member 30 having a first end and a second end, the second end of the tip member 30 being oppositely disposed relative to the first end, the tip member 30 including a first wall 33 disposed at the first end thereof, the first wall 33 defining a first aperture 31 configured to be in fluid communication with a nostril of a person; a cover member 20 disposed over a portion of the tip member 30, the cover member 20 including a second wall 23 overlapping at least a portion of the first wall 33 of the tip member 30, the second wall 23 defining a second aperture 24 configured to be in fluid communication with the nostril of the person and the first aperture 31 of the tip member 30, the cover member 20 configured to provide a suction seal with the nostril of the person; and a bulb member 40 coupled to the tip member 30, the bulb member 40 configured to create a vacuum force for removing mucus from the nostril of the person and through the first and second apertures 31, 24, the vacuum force being created as a result of the bulb member 40 being initially compressed by a user, and then the user gradually releasing the compression of the bulb member 40.

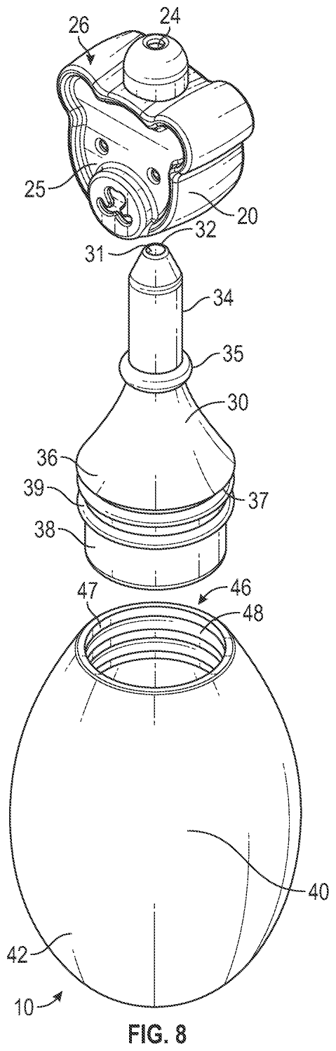

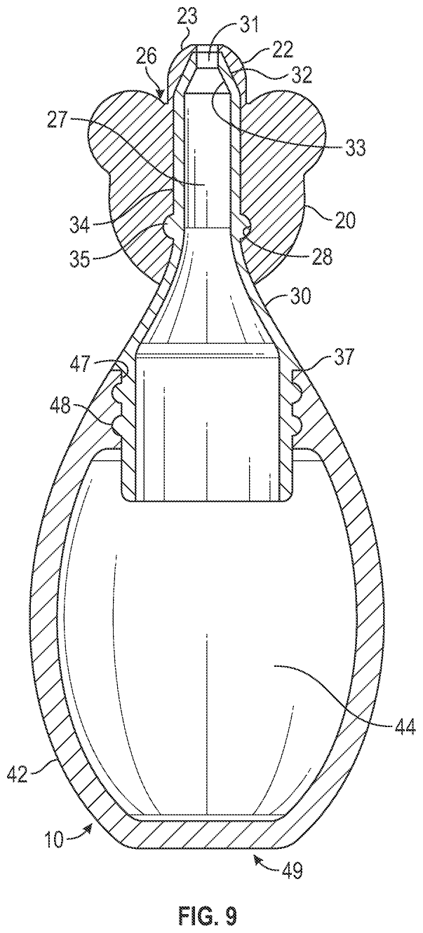

In the illustrative embodiment, referring again to FIGS. 1, 8, and 9, it can be seen that the tip member 30 further comprises a tapered distal end portion 32 at the first end thereof, and an attachment collar portion 38 at the second end thereof. The tapered distal end portion 32 of the tip member 30 includes the first wall 33 and is disposed inside the cover member 20 (see FIG. 9), and the attachment collar portion 38 of the tip member 30 is received within an opening 46 of the bulb member 40 so as to attach the tip member 30 to the bulb member 40 (see FIGS. 8 and 9). In the illustrative embodiment, as shown in FIGS. 8 and 9, the tapered distal end portion 32 of the tip member 30 has a frustoconical shape so as to form a converging nozzle at the end of the tip member 30. Also, in the illustrative embodiment, the attachment collar portion 38 has a cylindrical shape (see FIGS. 8 and 9).

With reference again to FIGS. 1, 8, and 9, it can be seen that, in the illustrative embodiment, the tip member 30 further comprises a stem portion 34 connecting the tapered distal end portion 32 at the first end thereof to the attachment collar portion 38 at the second end thereof. As shown in FIGS. 8 and 9, the stem portion 34 of the tip member 30 further comprises an annular protrusion 35 disposed thereon, and the cover member 20 further comprises a central passageway 27 with an annular groove 28 disposed in the central passageway 27. The annular protrusion 35 on the stem portion 34 of the tip member 30 matingly engages with the annular groove 28 in the central passageway 27 of the cover member 20 so as to securely attach the cover member 20 to the tip member 30.

In the illustrative embodiment of FIGS. 8 and 9, it can be seen that the stem portion 34 is connected to the attachment collar portion 38 by a frustoconical base portion 36. Also, in the illustrative embodiment, the attachment collar portion 38 of the tip member 30 comprises a plurality of annular protrusions 39 disposed thereon (see FIGS. 8 and 9), and the opening 46 of the bulb member 40 is circumscribed by a bounding wall 47 with a plurality of annular grooves 48 formed therein. As shown in the sectional view of FIG. 9, the plurality of annular protrusions 39 on the attachment collar portion 38 of the tip member 30 matingly engage with the plurality of annular grooves 48 in the bounding wall 47 of the bulb member 40 so as to securely attach the tip member 30 to the bulb member 40. In the illustrative embodiment, it can be seen that the tip member 30 defines a ledge 37 at the bottom of the frustoconical base portion 36 that rests on a top rim of the bulb member 40 when the tip member 30 is inserted into the bulb member 40.

Now, turning again to FIGS. 1, 8, and 9, the cover member 20 of the illustrative nasal aspirator 10 will be described in further detail. In the illustrative embodiment, it can be seen that the cover member 20 further comprises a hemispherical protruding portion 22 that includes the second wall 23. The hemispherical protruding portion 22 operates as a primary stop portion sized to limit the depth that the second wall 23 is able to be inserted into the nostril of the person. Also, in the illustrative embodiment, a surface portion of the hemispherical protruding portion 22 of the cover member 20 is configured to provide the suction seal with the nostril of the person.

Referring again to FIGS. 1, 8, and 9, in the illustrative embodiment, it can be seen that the cover member 20 further comprises a cover body portion 25 connected to the hemispherical protruding portion 22. The cover body portion 25 operates as a stabilizer when the hemispherical protruding portion 22 is being inserted into the nostril of the person (e.g., the cover body portion 25 can be rested on the mouth of the baby so as to stabilize the nasal aspirator 10 in place). Also, in the illustrative embodiment, the cover body portion 25 further operates as a secondary stop portion sized to prevent over-insertion of the nasal aspirator 10 into the nostril of the person (e.g., the top of the cover body portion 25 forms a stop surface 26 for preventing over-insertion of the nasal aspirator 10 into the nostril of the person).

In the illustrative embodiment of the nasal aspirator 10 (see FIGS. 1-3, 8, and 9), the cover body portion 25 is provided with ornamental features that resemble the head of a “teddy bear,” with the top of the bear head body portion 25 forming the secondary stop portion 26 or stop surface. However, various other ornamental features may be used. For example, in other embodiments, the cover body portion 25 may take on other suitable shapes, such as but not limited to, the head shapes of other animals. Alternatively, a flat stop surface without any ornamentation can be used.

Next, with reference to FIGS. 1 and 7-9, the bulb member 40 of the illustrative nasal aspirator 10 will be described in further detail. In the illustrative embodiment, the bulb member 40 comprises bulb body portion 42 with a generally flat bottom surface 49 so that the nasal aspirator 10 is capable of being positioned in an upright position on a support surface. As shown in FIGS. 1 and 7-9, the bulb body portion 42 of the bulb member 40 defines an internal cavity 44 that operates as an air chamber. In the illustrative embodiment, the internal cavity 44 of the bulb body portion 42 of the bulb member 40 has a volume of approximately 65 milliliters (65 ml), which is larger in size as compared to conventional aspirators so that increased suction is produced by the nasal aspirator 10.

In the illustrative embodiment, the tip member 30 of the nasal aspirator 10 is formed from a first polymeric material, and the cover member 20 and the bulb member 40 of the nasal aspirator 10 are formed from a second polymeric material. In the illustrative embodiment, the second polymeric material is more flexible than the first polymeric material. More specifically, in the illustrative embodiment, the first polymeric material forming the tip member 30 comprises a food-grade polypropylene homopolymer (PPH), and the second polymeric material forming the cover member 20 and the bulb member 40 comprises Food & Drug Administration (FDA)-approved, food-grade, virgin silicone. As such, in the illustrative embodiment, the first polymeric material forming the tip member 30 and the second polymeric material forming the cover member 20 and the bulb member 40 do not comprise latex, bisphenol A (BPA), or polyvinyl chloride (PVC) (i.e., the materials are latex-free, BPA-free, and PVC-free).

In the illustrative embodiment, the tip member 30, the cover member 20, and the bulb member 40 are capable of being dissembled from one another for easy cleaning of the nasal aspirator 10. As shown in the exploded view of FIG. 8, the tip member 30, the cover member 20, and the bulb member 40 are able to be separated from one another so that each of the components 20, 30, 40 are able to be separately cleaned with soap and water. Then, after the components 20, 30, 40 are cleaned, the tip member 30, the cover member 20, and the bulb member 40 are able to be easily reassembled with one another in a snap-fit type assembly.

Now, with reference to FIGS. 1-3, the manner in which the nasal aspirator 10 of the illustrative embodiment is used will be described. Initially, a portion of the hemispherical protruding portion 22 of the cover member 20 is inserted into the nostril of a person (e.g., the nostril of a baby). After which, a user creates a vacuum force by initially compressing the bulb member 40, and then gradually releasing the compression of the bulb member 40. The vacuum force created by the bulb member 40 extracts mucus from the nostril of the person, and pulls the mucus into the nasal aspirator 10 through the apertures 24, 31 so as to at least partially clear the nasal passage of the person.

While the nasal aspirator 10 is described for extracting mucus from the nostril of the person in the illustrative embodiment, it is to be understood that the aspirator may be used for many different applications requiring the generation of a suction force. Also, the innovative seal created by the compliant outer wall 23 disposed over the semi-rigid tip wall 33 of the aspirator 10 may be used in a variety of different applications with many different vacuum sources.

It is readily apparent that the illustrative nasal aspirator 10 offers numerous advantages. First, the nasal aspirator 10 is easy to disassemble and clean. Secondly, the nasal aspirator 10 includes primary and secondary stop portions 22, 26 for preventing the aspirator from being over-inserted into the nostril of the person. Thirdly, the nasal aspirator 10 forms an effective suction seal with the nostril of the person. Finally, the nasal aspirator 10 has a larger bulb size as compared to conventional aspirators so that increased suction is produced.

Advantageously, the aforedescribed bulb-suction aspirator 10 has three (3) detachable components 20, 30, 40 that can be easily pulled-apart and reassembled for effective cleaning. The suction bulb 40 can be squeezed by hand to produce a strong suction force for booger/mucus removal. In one or more illustrative embodiments, a portion of the hemispherical protruding portion 22 of the cover member 20 may be inserted into the nostril of a baby. The size of the hemispherical protruding portion 22 makes it unique and a perfect fit for a baby's nose. The converging nozzle distal end portion 32 of the tip member 30 is covered by a soft bear-head 25 with a hemispherical protruding portion 22 to cover the distal end portion 32 of the tip member 30. The bear head 25 prevents the distal end portion 32 from going too far into the nose, and stabilizes the aspirator 10 in place so that it is easy to use. The cover portion 20 also increases the power of the suction by providing a complete seal, and the soft silicone of the illustrative embodiment ensures that the aspirator 10 is comfortable on the nose. The bulb member 40 of the illustrative nasal aspirator 10 is larger, and thus creates more suction than conventional nasal aspirators.

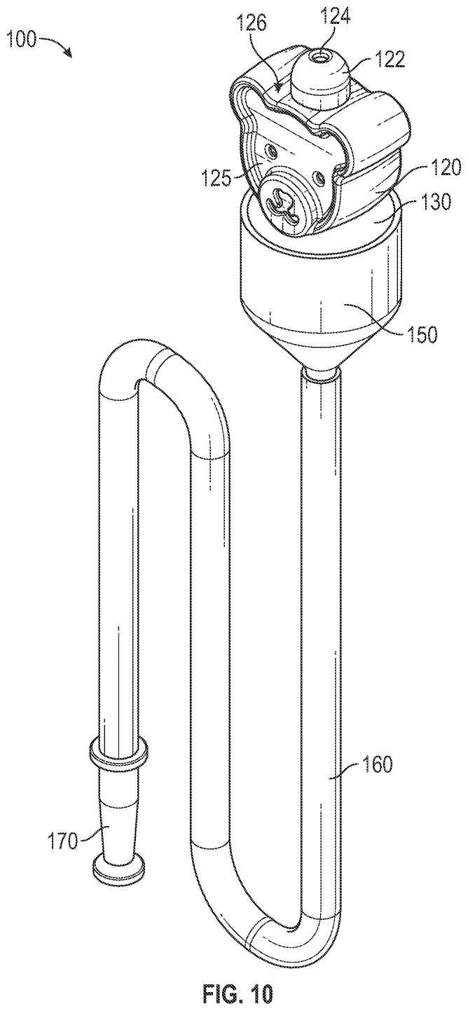

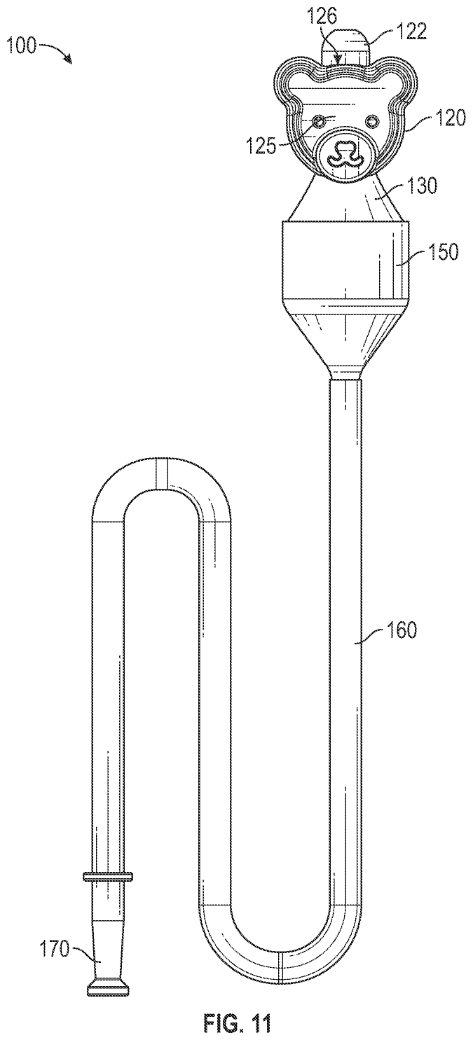

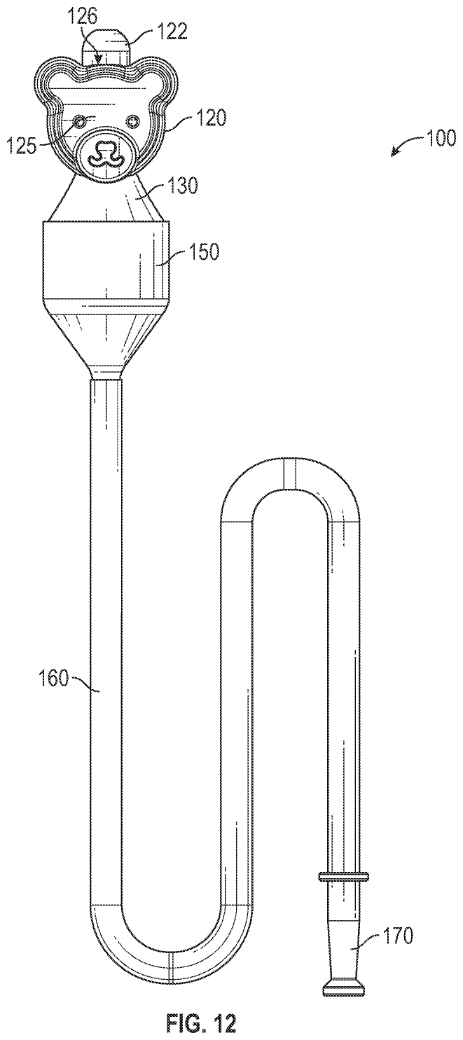

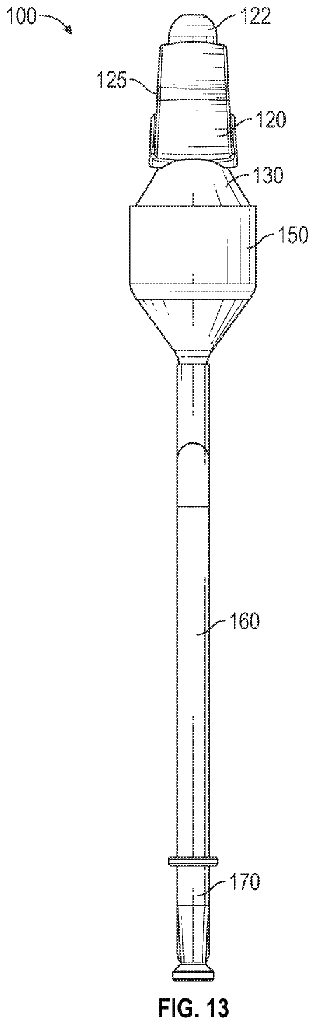

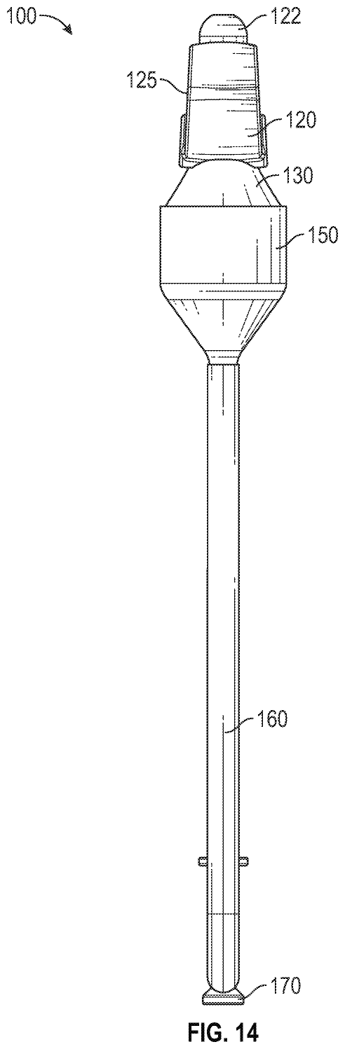

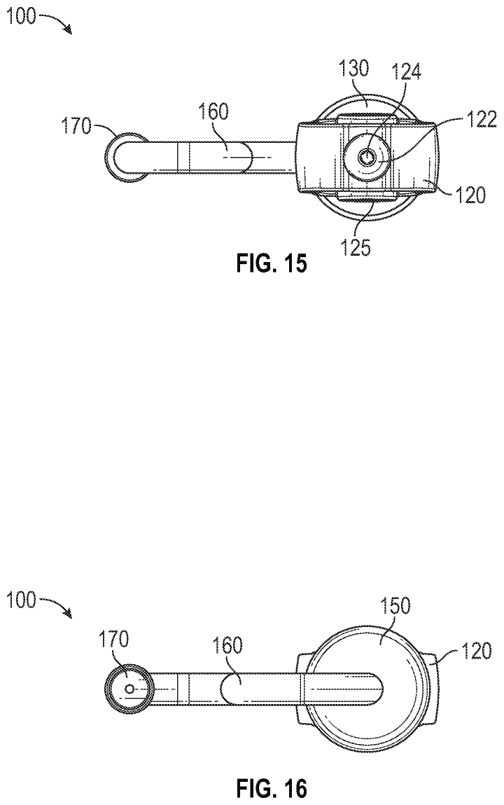

A second illustrative embodiment of a nasal device is seen generally at 100 in FIGS. 10-20. In the second illustrative embodiment, the nasal device is in a form of a mouth suction nasal aspirator 100. With initial reference to FIGS. 10, 17, and 20, it can be seen that the mouth suction nasal aspirator 100 generally comprises a tip member 130 having a first end and a second end, the second end of the tip member 130 being oppositely disposed relative to the first end, the tip member 130 including a first wall 133 disposed at the first end thereof, the first wall 133 defining a first aperture 131 configured to be in fluid communication with a nostril of a person; a cover member 120 disposed over a portion of the tip member 130, the cover member 120 including a second wall 123 overlapping at least a portion of the first wall 133 of the tip member 130, the second wall 123 defining a second aperture 124 configured to be in fluid communication with the nostril of the person and the first aperture 131 of the tip member 130, the cover member 120 configured to provide a suction seal with the nostril of the person; a connector member 150 that is attached to an attachment collar portion 138 of the tip member 130; a filter member 140 disposed in the connector member 150; and a mouthpiece 170 connected to the connector member 150 by means of a flexible tube 160. In this illustrative embodiment, the mouth suction aspirator 100 is configured to create a vacuum force for removing mucus from the nostril of the person (e.g., a baby) and through the first and second apertures 131, 124 when a user (e.g., a parent) places the mouthpiece 170 into his or her mouth and draws air through a distal end of the mouthpiece 170, the filter member 140 configured to trap mucus from the nostril of the person so as to prevent the mucus, viruses, and/or bacteria from entering the mouth of the user.

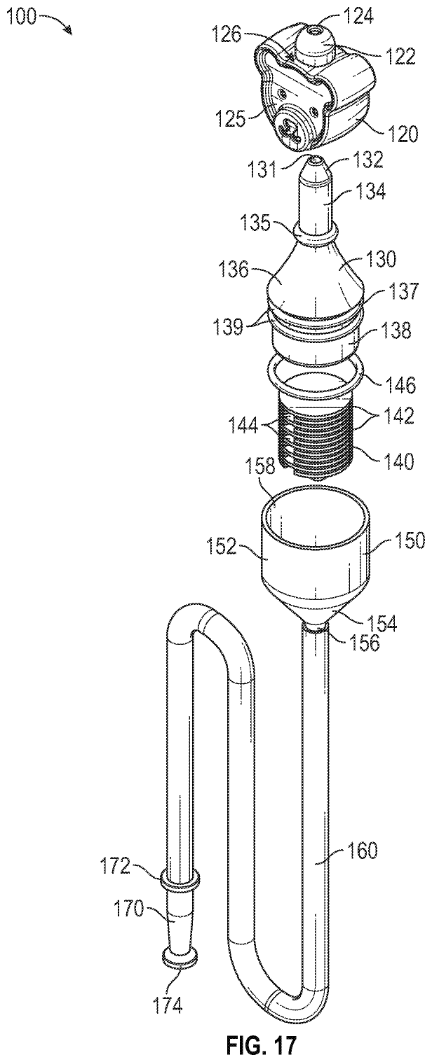

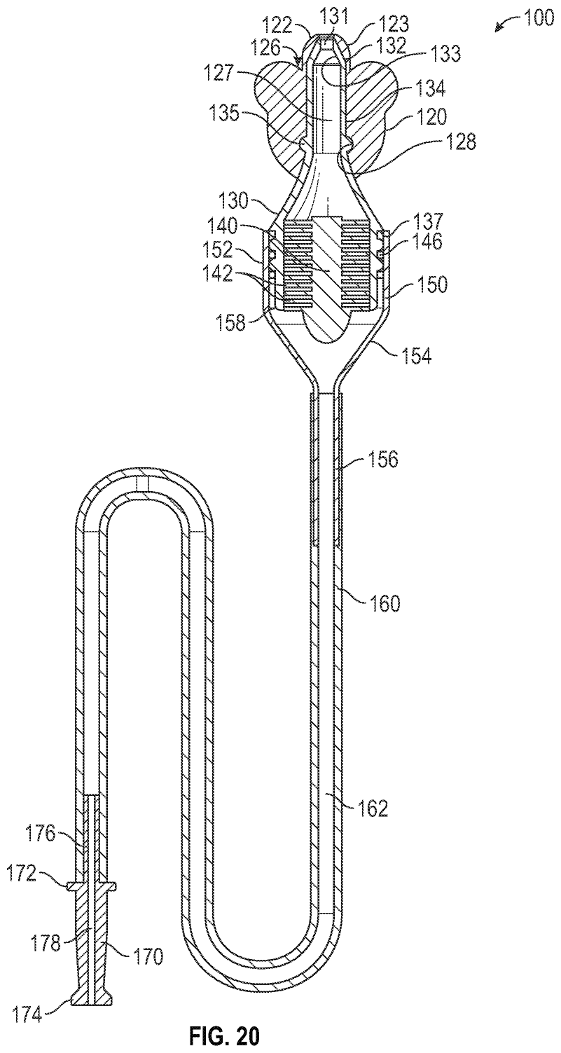

In the illustrative embodiment, referring again to FIGS. 10, 17, and 20, it can be seen that the tip member 130 further comprises a tapered distal end portion 132 at the first end thereof, and the attachment collar portion 138 at the second end thereof. The tapered distal end portion 132 of the tip member 130 includes the first wall 133 and is disposed inside the cover member 120 (see FIG. 20), and the attachment collar portion 138 of the tip member 130 is received within the cylindrical recess 158 of the connector member 150 so as to attach the tip member 130 to the connector member 150 (see FIGS. 17 and 20). In the illustrative embodiment, as shown in FIGS. 17 and 20, the tapered distal end portion 132 of the tip member 130 has a frustoconical shape so as to form a converging nozzle at the end of the tip member 130. Also, in the illustrative embodiment, the attachment collar portion 138 has a cylindrical shape (see FIGS. 17 and 20).

With reference again to FIGS. 10, 17, and 20, it can be seen that, in the illustrative embodiment, the tip member 130 further comprises a stem portion 134 connecting the tapered distal end portion 132 at the first end thereof to the attachment collar portion 138 at the second end thereof. As shown in FIGS. 17 and 20, the stem portion 134 of the tip member 130 further comprises an annular protrusion 135 disposed thereon, and the cover member 120 further comprises a central passageway 127 with an annular groove 128 disposed in the central passageway 127. The annular protrusion 135 on the stem portion 134 of the tip member 130 matingly engages with the annular groove 128 in the central passageway 127 of the cover member 120 so as to securely attach the cover member 120 to the tip member 130.

In the illustrative embodiment of FIGS. 17 and 20, it can be seen that the stem portion 134 is connected to the attachment collar portion 138 by a frustoconical base portion 136. Also, in the illustrative embodiment, the attachment collar portion 138 of the tip member 130 comprises a plurality of annular protrusions 139 disposed thereon (see FIGS. 17 and 20) with an elastomeric O-ring member 146 disposed between the annular protrusions 139. As shown in the sectional view of FIG. 20, the plurality of annular protrusions 139 on the attachment collar portion 138 of the tip member 130 and the elastomeric O-ring member 146 matingly engage with the interior surface of the cylindrical upper wall 152 of the connector member 150 so as to securely attach the tip member 130 to the connector member 150. In the illustrative embodiment, it can be seen that the tip member 130 defines a ledge 137 at the bottom of the frustoconical base portion 136 that rests on a top rim of the cylindrical upper wall 152 of the connector member 150 when the tip member 130 is inserted into the cylindrical recess 158 of the connector member 150. The elastomeric O-ring member 146 provides a generally airtight seal between the tip member 130 and the connector member 150 when the tip member 130 is inserted into the cylindrical recess 158 of the connector member 150.

Now, turning again to FIGS. 10, 17, and 20, the cover member 120 of the illustrative nasal aspirator 100 will be described in further detail. In the illustrative embodiment, it can be seen that the cover member 120 further comprises a hemispherical protruding portion 122 that includes the second wall 123. The hemispherical protruding portion 122 operates as a primary stop portion sized to limit the depth that the second wall 123 is able to be inserted into the nostril of the person. Also, in the illustrative embodiment, a surface portion of the hemispherical protruding portion 122 of the cover member 120 is configured to provide the suction seal with the nostril of the person.

Referring again to FIGS. 10, 17, and 20, in the illustrative embodiment, it can be seen that the cover member 120 further comprises a cover body portion 125 connected to the hemispherical protruding portion 122. The cover body portion 125 operates as a stabilizer when the hemispherical protruding portion 122 is being inserted into the nostril of the person (e.g., the cover body portion 125 can be rested on the mouth of the baby so as to stabilize the nasal aspirator 100 in place). Also, in the illustrative embodiment, the cover body portion 125 further operates as a secondary stop portion sized to prevent over-insertion of the nasal aspirator 100 into the nostril of the person (e.g., the top of the cover body portion 125 forms a stop surface 126 for preventing over-insertion of the nasal aspirator 100 into the nostril of the person).

In the illustrative embodiment of the nasal aspirator 100 (see FIGS. 10-12, 17, and 20), the cover body portion 125 is provided with ornamental features that resemble the head of a “teddy bear,” with the top of the bear head body portion 125 forming the secondary stop portion 126 or stop surface. However, various other ornamental features may be used. For example, in other embodiments, the cover body portion 125 may take on other suitable shapes, such as but not limited to, the head shapes of other animals. Alternatively, a flat stop surface without any ornamentation can be used.

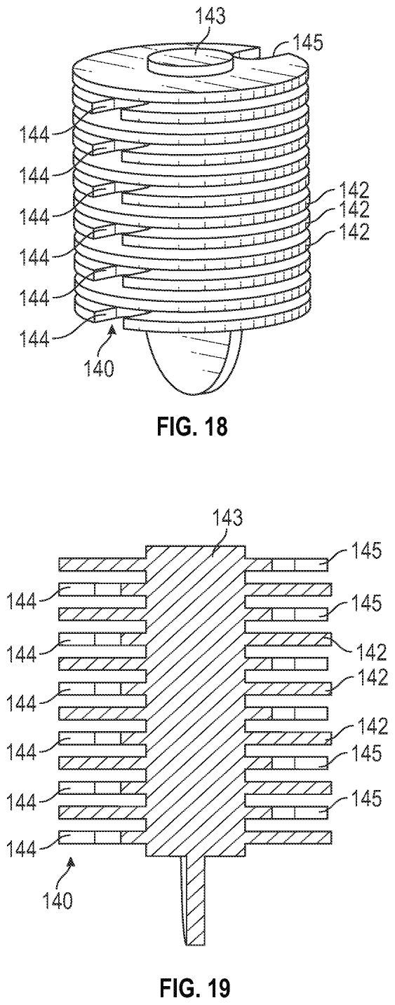

Next, with reference to FIGS. 17-19, the filter member 140 of the illustrative mouth suction nasal aspirator 100 will be described in further detail. In the illustrative embodiment, the filter member 140 comprises a plurality of spaced-apart disks 142 that are mounted to a central core portion 143 (see FIGS. 18 and 19). As shown in FIGS. 18 and 19, each successive spaced-apart disk 142 is provided with a notch that is disposed on an opposite side relative to the notches of the disks 142 above and below that disk 142. For example, referring to FIGS. 18 and 19, it can be seen that the bottommost disk 142 is provided with a notch 144 formed therethrough, and the disk 142 above the bottommost disk 142 is provided with an oppositely disposed notch 145 formed therethrough (i.e., the notch 145 is spaced 180 degrees apart from the notch 144 in the bottommost disk 142). This offset notch pattern continues along the entire longitudinal axis of the filter member 140 so as to create a zigzag or serpentine air path for trapping the mucus from the nostril of the person inside the filter member 140.

Now, referring again to FIGS. 17 and 20, the connector member 150, the flexible tube 160, and the mouthpiece 170 of the illustrative mouth suction nasal aspirator 100 will be explained in further detail. As shown in these figures, the connector member 150 further comprises a frustoconical base portion 154 that is connected to the bottom of the cylindrical upper wall 152 of the connector member 150. The connector member 150 further comprises a tube stem portion 156 that is connected to the bottom of the frustoconical base portion 154. As best shown in FIG. 20, the end portion of the flexible tube 160 is fitted over the tube stem portion 156 of the connector member 150 so as to connect the flexible tube 160 to the connector member 150 in a generally airtight manner. The flexible tube 160 defines an interior passageway 162 for conveying suction air drawn through the tube 160 by the user. Turning again to FIGS. 17 and 20, it can be seen that the mouthpiece 170 of the illustrative mouth suction nasal aspirator 100 comprises a proximal end portion 172, an oppositely disposed distal end portion 174, a tube stem portion 176 extending from the proximal end portion 172 of the mouthpiece 170, and an internal passageway 178 for conveying suction air drawn through the mouthpiece 170 by the user. The flexible tube 160 is fitted over the tube stem portion 176 so as to connect the flexible tube 160 to the mouthpiece 170 in a generally airtight manner.

In the illustrative embodiment, the tip member 130 of the nasal aspirator 100 is formed from a first polymeric material, and the cover member 120 of the nasal aspirator 100 is formed from a second polymeric material. In the illustrative embodiment, the second polymeric material is more flexible than the first polymeric material. More specifically, in the illustrative embodiment, the first polymeric material forming the tip member 130 comprises a food-grade polypropylene homopolymer (PPH), and the second polymeric material forming the cover member 120 comprises Food & Drug Administration (FDA)-approved, food-grade, virgin silicone. As such, in the illustrative embodiment, the first polymeric material forming the tip member 130 and the second polymeric material forming the cover member 120 do not comprise latex, bisphenol A (BPA), or polyvinyl chloride (PVC) (i.e., the materials are latex-free, BPA-free, and PVC-free).

In the illustrative embodiment, the tip member 130, the cover member 120, the connector member 150, and the filter member 140 are capable of being dissembled from one another for easy cleaning of the nasal aspirator 100. As shown in the exploded view of FIG. 17, the tip member 130, the cover member 120, the connector member 150, and the filter member 140 are able to be separated from one another so that each of the components 120, 130, 140, 150 are able to be separately cleaned with soap and water. Then, after the components 120, 130, 140, 150 are cleaned, the tip member 130, the cover member 120, the connector member 150, and the filter member 140 are able to be easily reassembled with one another in a snap-fit type assembly. In one or more embodiments, the other components of the mouth suction nasal aspirator 100 may also be capable of being dissembled from one another for easy cleaning of the aspirator 100.

Now, with reference to FIGS. 10-12, the manner in which the nasal aspirator 100 of the illustrative embodiment is used will be described. Initially, a portion of the hemispherical protruding portion 122 of the cover member 120 is inserted into the nostril of a person (e.g., the nostril of a baby). After which, a user (e.g., a parent of the baby) creates a vacuum force by sucking on the mouthpiece 170 of the nasal aspirator 100 with his or her mouth. The vacuum force created by the mouth suction of the user extracts mucus from the nostril of the person, and pulls the mucus into the nasal aspirator 100 through the apertures 124, 131 so as to at least partially clear the nasal passage of the person. In the illustrative embodiment, the filter member 140 prevents the mucus from going into the user's mouth and acts as a barrier for viruses and bacteria. The filter member 140 acts as a barrier for trapping bacteria and virus using the zigzag configuration described above, which traps the bacteria/virus from moving beyond the filter member 140 of the aspirator 100. The filter member 140 is useable and durable. Also, as described above, filter member 140 may be completely removed from the connector member 150 for cleaning once the tip member 130 has been detached from the connector member 150 by disengaging the friction fit between the components 130, 150. Alternatively, the tip member 130 and the connector member 150 may be removably attached to, and detached from one another using a screw-on connection with corresponding threads on the tip member 130 and the connector member 150.

While the mouth suction nasal aspirator 100 is described for extracting mucus from the nostril of the person in the illustrative embodiment, it is to be understood that the aspirator may be used for many different applications requiring the generation of a suction force. Also, the innovative seal created by the compliant outer wall 123 disposed over the semi-rigid tip wall 133 of the aspirator 100 may be used in a variety of different applications with many different vacuum sources.

It is readily apparent that the illustrative mouth suction nasal aspirator 100 offers numerous advantages. First, the nasal aspirator 100 is easy to disassemble and clean. Secondly, the nasal aspirator 100 includes primary and secondary stop portions 122, 126 for preventing the aspirator from being over-inserted into the nostril of the person. Finally, the nasal aspirator 100 forms an effective suction seal with the nostril of the person.

Advantageously, the aforedescribed mouth suction nasal aspirator 100 comprises a cover member 120 that acts as a barrier between the tip member 130 and the baby's nostril. This allows the device 100 to be comfortable for baby and allows parents to approach without fear of hurting the baby. Moreover, cover member 120 of the nasal aspirator 100 also acts as a stabilizer for the parent using the device 100. The design of the cover member 120 provides an additional layer of safety for the baby when the device 100 is being used. Furthermore, the soft large cover member 120 prevents the device 100 from entering the baby's nose too far. The cover member 120 also produces a great seal to be able to suction out the mucus.

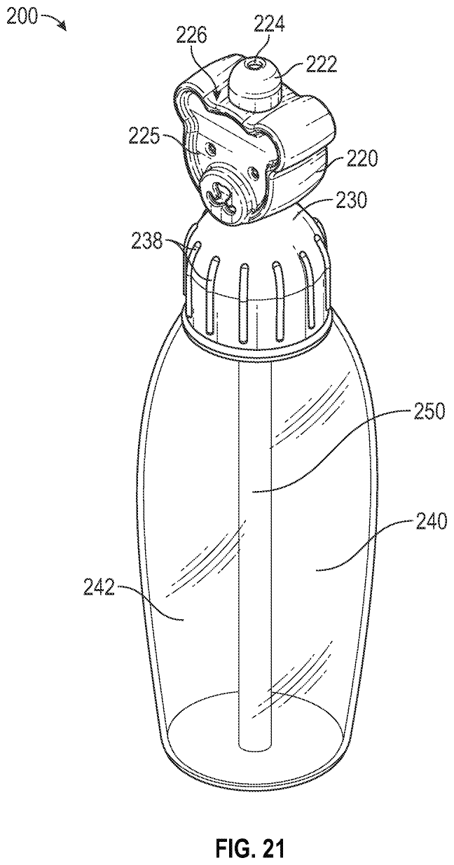

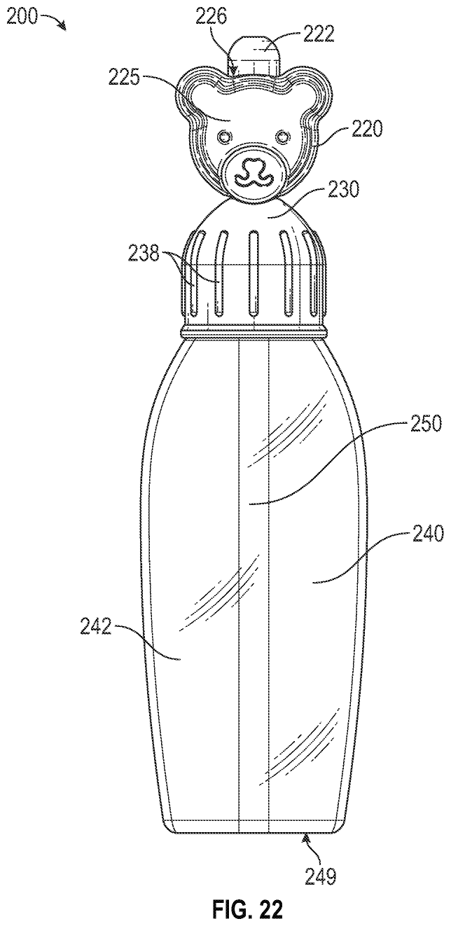

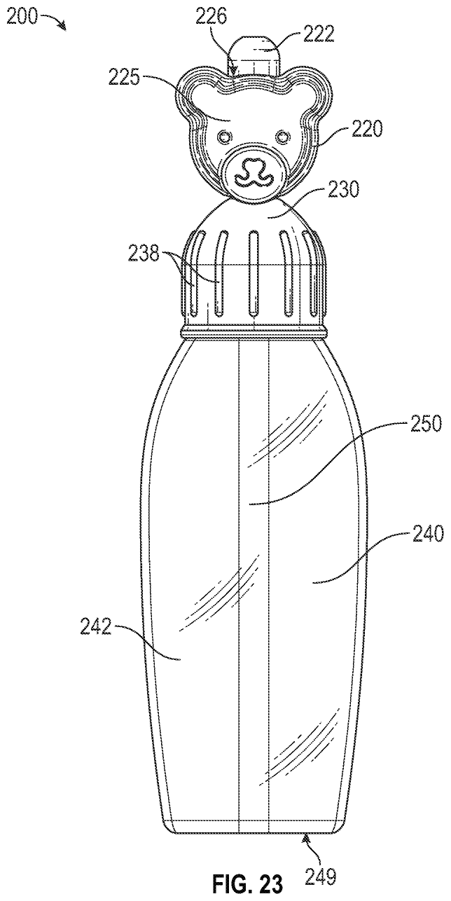

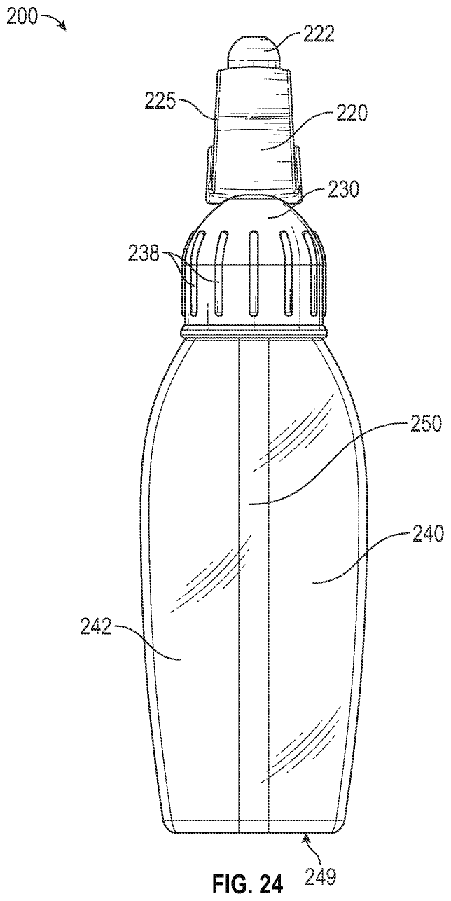

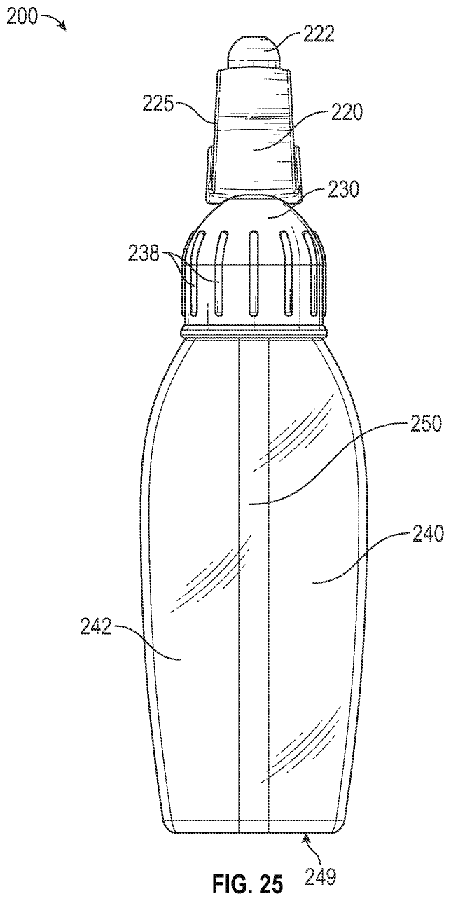

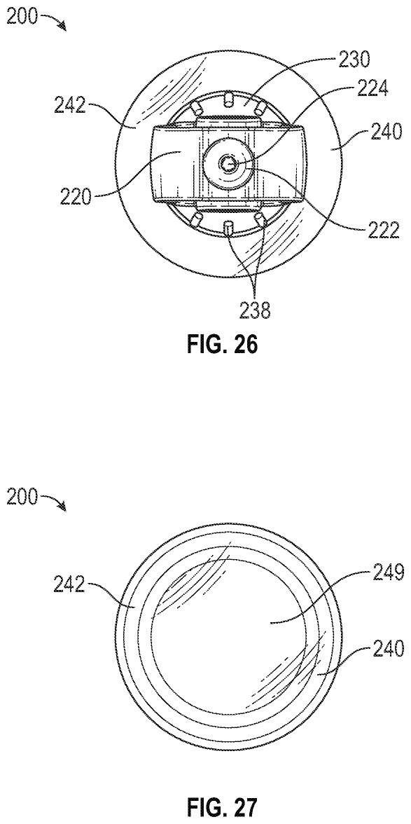

A third illustrative embodiment of a nasal device is seen generally at 200 in FIGS. 21-29. In the third illustrative embodiment, the nasal device is in a form of a nasal irrigation bottle 200. With initial reference to FIGS. 21, 28, and 29, it can be seen that the nasal irrigation bottle 200 generally comprises a tip cap member 230 having a first end and a second end, the second end of the tip cap member 230 being oppositely disposed relative to the first end, the tip cap member 230 including a first wall 233 disposed at the first end thereof, the first wall 233 defining a first aperture 231 configured to be in fluid communication with a nostril of a person; a cover member 220 disposed over a portion of the tip cap member 230, the cover member 220 including a second wall 223 overlapping at least a portion of the first wall 233 of the tip cap member 230, the second wall 223 defining a second aperture 224 configured to be in fluid communication with the nostril of the person and the first aperture 231 of the tip cap member 230, the cover member 220 configured to provide a seal with the nostril of the person; and a bottle member 240 coupled to the tip cap member 230, the bottle member 240 configured to hold a liquid solution for being expelled through the first and second apertures 231, 224 and into the nostril of the person.

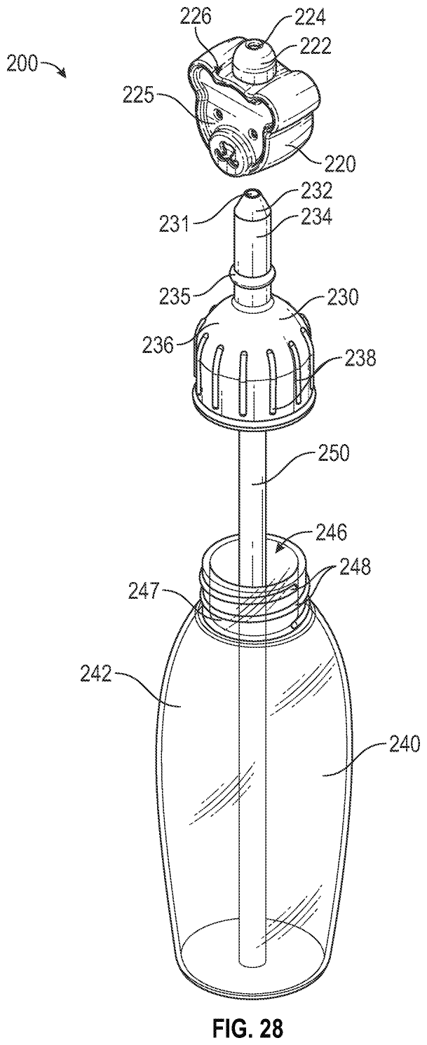

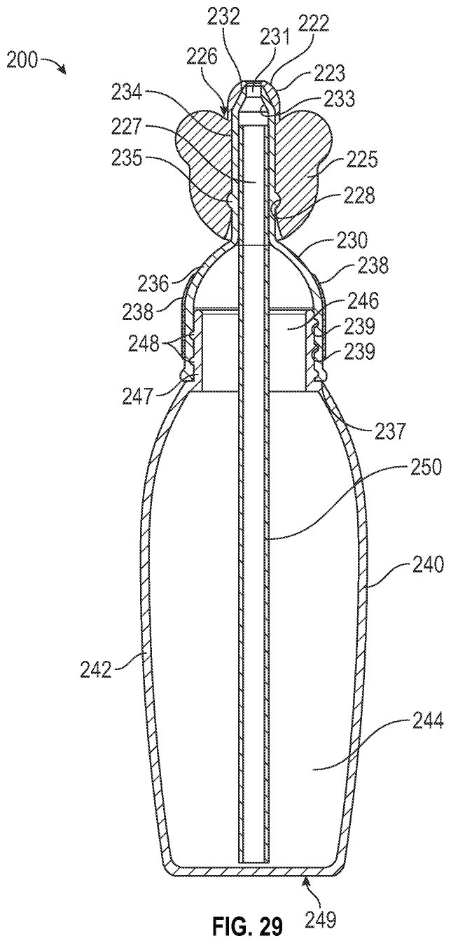

In the illustrative embodiment, referring again to FIGS. 21, 28, and 29, it can be seen that the tip cap member 230 further comprises a tapered distal end portion 232 at the first end thereof, and a hemispherical base portion 236 at the second end thereof. The tapered distal end portion 232 of the tip cap member 230 includes the first wall 233 and is disposed inside the cover member 220 (see FIG. 29), and the hemispherical base portion 236 of the tip cap member 230 is fitted over a top opening 246 of the bottle member 240 so as to function as a cap for the bottle member 240 (see FIGS. 28 and 29). In the illustrative embodiment, as shown in FIGS. 28 and 29, the tapered distal end portion 232 of the tip cap member 230 has a frustoconical shape so as to form a converging nozzle at the end of the tip cap member 230. Also, in the illustrative embodiment, the hemispherical base portion 236 of the tip cap member 230 has a hemispherical or dome shape (see FIGS. 28 and 29).

With reference again to FIGS. 21, 28, and 29, it can be seen that, in the illustrative embodiment, the tip cap member 230 further comprises a stem portion 234 connecting the tapered distal end portion 232 at the first end thereof to the hemispherical base portion 236 at the second end thereof. As shown in FIGS. 28 and 29, the stem portion 234 of the tip cap member 230 further comprises an annular protrusion 235 disposed thereon, and the cover member 220 further comprises a central passageway 227 with an annular groove 228 disposed in the central passageway 227. The annular protrusion 235 on the stem portion 234 of the tip cap member 230 matingly engages with the annular groove 228 in the central passageway 227 of the cover member 220 so as to securely attach the cover member 220 to the tip cap member 230.

In the illustrative embodiment of FIGS. 28 and 29, it can be seen that the stem portion 234 is connected to the top of the tip cap member 230. Also, in the illustrative embodiment, the interior surface of the hemispherical base portion 236 of the tip cap member 230 comprises a plurality of internal threads 239 disposed thereon (see FIG. 29), and the top collar 247 of the bottle member 240 comprises a plurality of corresponding external threads 248 disposed thereon. As shown in the sectional view of FIG. 29, the plurality of external threads 248 on the top collar 247 of the bottle member 240 matingly engage with the plurality of internal threads 239 of tip cap member 230 so as to securely attach the tip cap member 230 to the bottle member 240 in a removable manner. In the illustrative embodiment, it can be seen that the tip cap member 230 defines a ledge 237 at the bottom of the hemispherical base portion 236 that rests on a ledge at the bottom of the bottle top collar 247 when the tip cap member 230 is attached to the bottle member 240. Referring again to FIGS. 21 and 28, it can be seen that the hemispherical base portion 236 of the tip cap member 230 comprises a plurality of vertically extending gripping protrusions 238 spaced apart about the periphery thereof so as to facilitate a user's grip on the tip cap member 230 when the user is attaching or detaching the tip cap member 230 from the bottle member 240.

Now, turning again to FIGS. 21, 28, and 29, the cover member 220 of the illustrative nasal irrigation bottle 200 will be described in further detail. In the illustrative embodiment, it can be seen that the cover member 220 further comprises a hemispherical protruding portion 222 that includes the second wall 223. The hemispherical protruding portion 222 operates as a primary stop portion sized to limit the depth that the second wall 223 is able to be inserted into the nostril of the person. Also, in the illustrative embodiment, a surface portion of the hemispherical protruding portion 222 of the cover member 220 is configured to provide the seal with the nostril of the person.

Referring again to FIGS. 21, 28, and 29, in the illustrative embodiment, it can be seen that the cover member 220 further comprises a cover body portion 225 connected to the hemispherical protruding portion 222. The cover body portion 225 operates as a stabilizer when the hemispherical protruding portion 222 is being inserted into the nostril of the person (e.g., the cover body portion 225 can be rested on the mouth of the baby so as to stabilize the nasal irrigation bottle 200 in place). Also, in the illustrative embodiment, the cover body portion 225 further operates as a secondary stop portion sized to prevent over-insertion of the nasal irrigation bottle 200 into the nostril of the person (e.g., the top of the cover body portion 225 forms a stop surface 226 for preventing over-insertion of the nasal irrigation bottle 200 into the nostril of the person).

In the illustrative embodiment of the nasal irrigation bottle 200 (see FIGS. 21-23, 28, and 29), the cover body portion 225 is provided with ornamental features that resemble the head of a “teddy bear,” with the top of the bear head body portion 225 forming the secondary stop portion 226 or stop surface. However, various other ornamental features may be used. For example, in other embodiments, the cover body portion 225 may take on other suitable shapes, such as but not limited to, the head shapes of other animals. Alternatively, a flat stop surface without any ornamentation can be used.

Next, with reference to FIGS. 21 and 27-29, the bottle member 240 of the illustrative nasal irrigation bottle 200 will be described in further detail. In the illustrative embodiment, the bottle member 240 comprises bottle body portion 242 with a generally flat bottom surface 249 so that the nasal irrigation bottle 200 is capable of being positioned in an upright position on a support surface (e.g., on a table). As shown in FIGS. 21 and 27-29, the bottle body portion 242 of the bottle member 240 defines an internal cavity 244 that holds the liquid solution (e.g., a saline solution) configured to be expelled through the first and second apertures 231, 224 and into the nostril of the person.

Referring again to FIGS. 21, 28, and 29, in the illustrative embodiment, it can be seen that the nasal irrigation bottle 200 may further comprise a straw member 250 extending from the tip cap member 230 to a bottom of the bottle member 240 so as to extract the liquid solution (e.g., the saline solution) from the bottom of the bottle member 240.

In the illustrative embodiment, the tip cap member 230 of the nasal irrigation bottle 200 is formed from a first polymeric material, and the cover member 220 and the bottle member 240 of the nasal irrigation bottle 200 are formed from a second polymeric material. In the illustrative embodiment, the second polymeric material is more flexible than the first polymeric material. More specifically, in the illustrative embodiment, the first polymeric material forming the tip cap member 230 comprises a food-grade polypropylene homopolymer (PPH), and the second polymeric material forming the cover member 220 and the bottle member 240 comprises Food & Drug Administration (FDA)-approved, food-grade, virgin silicone. As such, in the illustrative embodiment, the first polymeric material forming the tip cap member 230 and the second polymeric material forming the cover member 220 and the bottle member 240 do not comprise latex, bisphenol A (BPA), or polyvinyl chloride (PVC) (i.e., the materials are latex-free, BPA-free, and PVC-free).

In the illustrative embodiment, the tip cap member 230, the cover member 220, and the bottle member 240 are capable of being dissembled from one another for easy cleaning of the nasal irrigation bottle 200. As shown in the exploded view of FIG. 28, the tip cap member 230, the cover member 220, and the bottle member 240 are able to be separated from one another so that each of the components 220, 230, 240 are able to be separately cleaned with soap and water. Then, after the components 220, 230, 240 are cleaned, the tip cap member 230, the cover member 220, and the bottle member 240 are able to be easily reassembled with one another. In one or more embodiments, the other components of the nasal irrigation bottle 200 may also be capable of being dissembled from one another for easy cleaning of the bottle 200.

Now, with reference to FIGS. 21-23, the manner in which the nasal irrigation bottle 200 of the illustrative embodiment is used will be described. Initially, a portion of the hemispherical protruding portion 222 of the cover member 220 is inserted into the nostril of a person (e.g., the nostril of a baby). After which, a user (e.g., a parent of the baby) expels the liquid solution (e.g., saline solution) from the bottle 240 by compressing the bottle body portion 242 of the bottle member 240 to provide nasal irrigation for the person.

While the nasal irrigation bottle 200 is described for providing nasal irrigation for the person in the illustrative embodiment, it is to be understood that the bottle 200 may be used for many different applications requiring the dispensing of a liquid solution. Also, the innovative seal created by the compliant outer wall 223 disposed over the semi-rigid tip wall 233 of the bottle 200 may be used in a variety of different applications with many different dispensing bottles.

It is readily apparent that the illustrative nasal irrigation bottle 200 offers numerous advantages. First, the nasal irrigation bottle 200 is easy to disassemble and clean. Secondly, the nasal irrigation bottle 200 includes primary and secondary stop portions 222, 226 for preventing the bottle 200 from being over-inserted into the nostril of the person. Finally, the irrigation bottle 200 forms an effective suction seal with the nostril of the person.

Advantageously, the aforedescribed nasal irrigation bottle 200 has three (3) detachable components 220, 230, 240 that can be disassembled and reassembled for effective cleaning. The nasal irrigation bottle 200 comprises a cover member 220 that acts as a barrier between the tip cap member 230 and the baby's nostril. This allows the device 200 to be comfortable for baby and allows parents to approach without fear of hurting the baby. Moreover, cover member 220 of the nasal irrigation bottle 200 also acts as a stabilizer for the parent using the device 200. The design of the cover member 220 provides an additional layer of safety for the baby when the device 200 is being used. In the illustrative embodiment, the application method is designed for the bottle member 240 to be squeezed expelling a saline solution (0.09% NaCl) into the baby's nostril. The cover member 220 using a silicone material creates a seal with baby's nostril, causing a rush of fluid to be rushed into the baby's nose and opening up the breathing pathways. This method will be easy to use, comfortable and smooth—flowing saline nasal irrigation for the baby. In the illustrative embodiment, the design of the bottle member 240 can mimic the aspirator bulb design only bigger in size to allow the volume needed for nasal irrigation. In the illustrative embodiment, the silicone material used for the bottle member 240 is safer than plastic, PABA free and anti-bacterial.

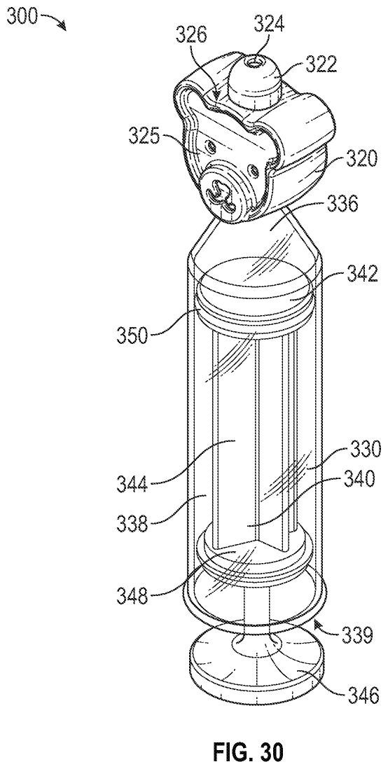

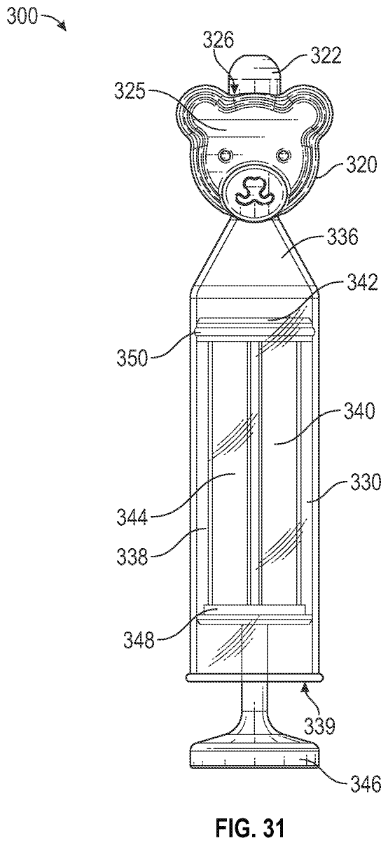

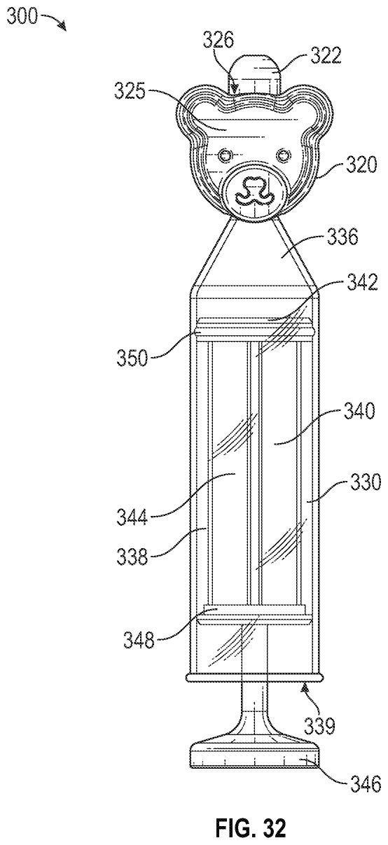

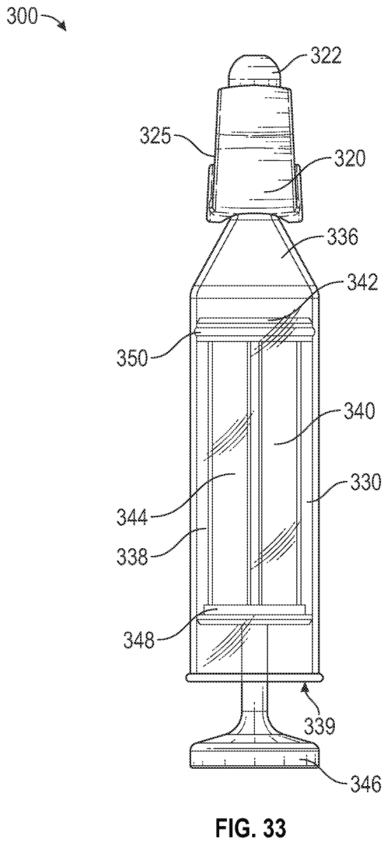

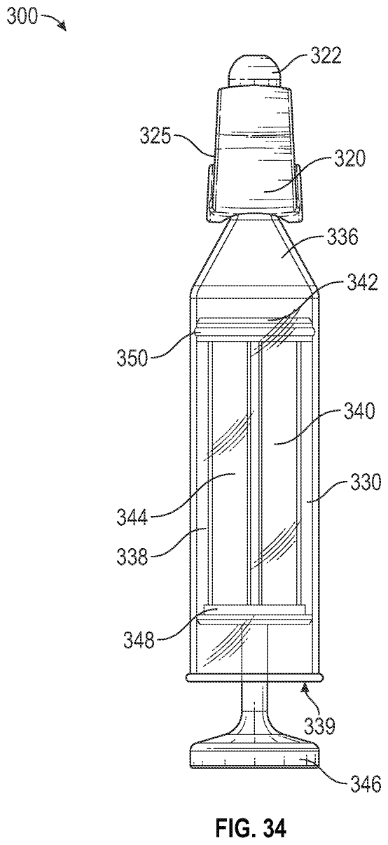

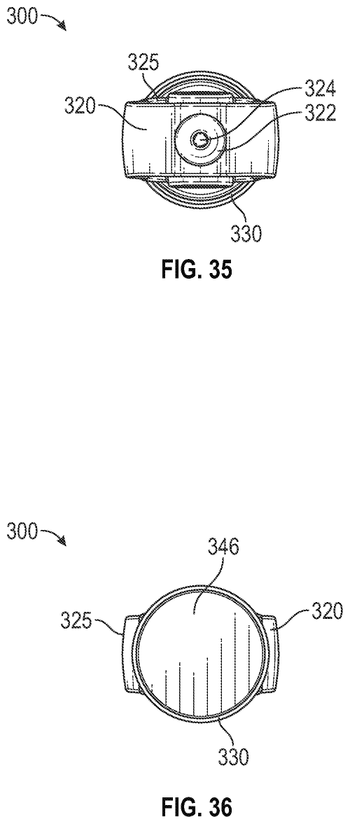

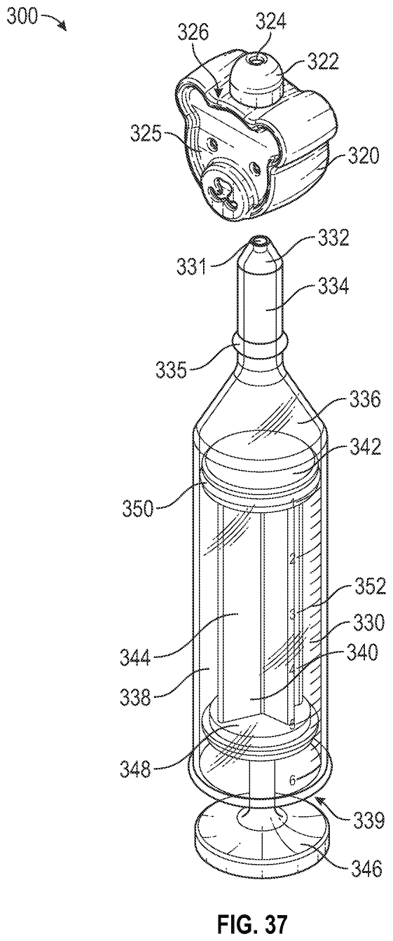

A fourth illustrative embodiment of a nasal device is seen generally at 300 in FIGS. 30-38. In the fourth illustrative embodiment, the nasal device is in a form of a nasal irrigation syringe 300. With initial reference to FIGS. 30, 37, and 38, it can be seen that the nasal irrigation syringe 300 generally comprises a syringe body 330 with a tip member having a first end and a second end, the second end of the tip member being oppositely disposed relative to the first end, the tip member including a first wall 333 disposed at the first end thereof, the first wall 333 defining a first aperture 331 configured to be in fluid communication with a nostril of a person; a cover member 320 disposed over a portion of the tip member of the syringe body 330, the cover member 320 including a second wall 323 overlapping at least a portion of the first wall 333 of the tip member, the second wall 323 defining a second aperture 324 configured to be in fluid communication with the nostril of the person and the first aperture 331 of the tip member of the syringe body 330, the cover member 320 configured to provide a seal with the nostril of the person; and a plunger assembly 340 slidably received within the syringe body 330, the plunger assembly 340 configured to be slidably displaced in a longitudinal direction of the syringe body 330, and the plunger assembly 340 configured to discharge the liquid solution from the nasal irrigation syringe 300 through the first and second apertures 331, 324, and into the nostril of the person when a force is applied to an end of the plunger assembly 340 by a user. As shown in FIGS. 30 and 37, the syringe body 330 comprises a fluid reservoir 338 for holding the liquid solution.

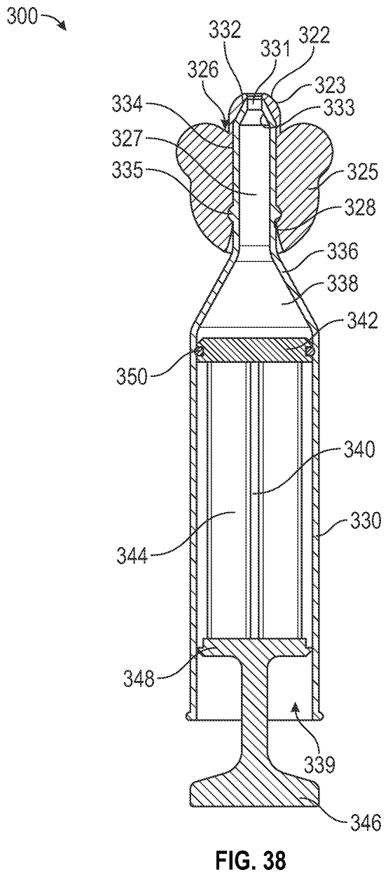

In the illustrative embodiment, referring again to FIGS. 30, 37, and 38, it can be seen that the tip member of the syringe body 330 further comprises a tapered distal end portion 332 at the first end thereof, and a frustoconical base portion 336 at the second end thereof. The tapered distal end portion 332 of the tip member includes the first wall 333 and is disposed inside the cover member 320 (see FIG. 38), and the frustoconical base portion 336 of the tip member is attached to the main cylindrical portion of the syringe body 330 (see FIGS. 30 and 37). In the illustrative embodiment, as shown in FIGS. 37 and 38, the tapered distal end portion 332 of the tip member has a frustoconical shape so as to form a converging nozzle at the end of the tip member of the syringe body 330. Also, in the illustrative embodiment, the frustoconical base portion 336 of the tip member has a frustoconical shape (see FIGS. 30 and 37).

With reference again to FIGS. 30, 37, and 38, it can be seen that, in the illustrative embodiment, the tip member of the syringe body 330 further comprises a stem portion 334 connecting the tapered distal end portion 332 at the first end thereof to the frustoconical base portion 336 at the second end thereof. As shown in FIGS. 30, 37, and 38, the stem portion 334 of the tip member further comprises an annular protrusion 335 disposed thereon, and the cover member 320 further comprises a central passageway 327 with an annular groove 328 disposed in the central passageway 327. The annular protrusion 335 on the stem portion 334 of the tip member matingly engages with the annular groove 328 in the central passageway 327 of the cover member 320 so as to securely attach the cover member 320 to the tip member of the syringe body 330.

Now, turning again to FIGS. 30, 37, and 38, the cover member 320 of the illustrative nasal irrigation syringe 300 will be described in further detail. In the illustrative embodiment, it can be seen that the cover member 320 further comprises a hemispherical protruding portion 322 that includes the second wall 323. The hemispherical protruding portion 322 operates as a primary stop portion sized to limit the depth that the second wall 323 is able to be inserted into the nostril of the person. Also, in the illustrative embodiment, a surface portion of the hemispherical protruding portion 322 of the cover member 320 is configured to provide the seal with the nostril of the person.

Referring again to FIGS. 30, 37, and 38, in the illustrative embodiment, it can be seen that the cover member 320 further comprises a cover body portion 325 connected to the hemispherical protruding portion 322. The cover body portion 325 operates as a stabilizer when the hemispherical protruding portion 322 is being inserted into the nostril of the person (e.g., the cover body portion 325 can be rested on the mouth of the baby so as to stabilize the nasal irrigation syringe 300 in place). Also, in the illustrative embodiment, the cover body portion 325 further operates as a secondary stop portion sized to prevent over-insertion of the nasal irrigation syringe 300 into the nostril of the person (e.g., the top of the cover body portion 325 forms a stop surface 326 for preventing over-insertion of the nasal irrigation syringe 300 into the nostril of the person).

In the illustrative embodiment of the nasal irrigation syringe 300 (see FIGS. 30-32, 37, and 38), the cover body portion 325 is provided with ornamental features that resemble the head of a “teddy bear,” with the top of the bear head body portion 325 forming the secondary stop portion 326 or stop surface. However, various other ornamental features may be used. For example, in other embodiments, the cover body portion 325 may take on other suitable shapes, such as but not limited to, the head shapes of other animals. Alternatively, a flat stop surface without any ornamentation can be used.

Next, with reference to FIGS. 30, 31, 37, and 38, the plunger assembly 340 of the illustrative nasal irrigation syringe 300 will be described in further detail. In the illustrative embodiment, the plunger assembly 340 comprises a plunger head portion 342, a plunger shaft 344 having a cross-sectional profile, and a plunger handle portion 346. The plunger handle portion 346 of the plunger assembly 340 is configured to receive a force applied by a user to discharge the liquid solution (e.g., saline solution) from the nasal irrigation syringe 300. In the illustrative embodiment, the plunger shaft 344 has a cross-sectional profile (e.g., cross-type or cross-member as shown in FIGS. 30 and 37). In an alternative embodiment, the plunger shaft 344 can have a circular cross-sectional profile. Referring again to FIGS. 30, 37, and 38, it can be seen that the plunger shaft 344 of the plunger assembly 340 further comprises an upper disk portion 348 attached to the plunger handle portion 346 and an elastomeric O-ring 350 disposed in a groove of the plunger head portion 342. The upper disk portion 348 of the plunger shaft 344 helps to stabilize the linear sliding motion of the plunger assembly 340. The elastomeric O-ring 350 creates a sealing engagement between the plunger head portion 342 and the circular sidewall of the syringe body 330 (i.e., the circular sidewall which circumscribes the fluid reservoir 338) so as to substantially prevent fluid leakage from the fluid reservoir 338. As shown in FIGS. 30, 37, and 38, the syringe body 330 is provided with an open end 339 through which the plunger assembly 340 is inserted.

Referring again to FIG. 37, in the illustrative embodiment, it can be seen that the syringe body 330 of the nasal irrigation syringe 300 may be provided with spaced-apart graduated marks 352 on the side of the syringe body 330 in order to indicate the fluid volume inside the syringe body 330 so that accurate volumetric quantities of the liquid solution (e.g., saline solution) may be dispensed using the nasal irrigation syringe 300.

In the illustrative embodiment, the syringe body 330 of the nasal irrigation syringe 300 is formed from a first polymeric material, and the cover member 320 of the nasal irrigation syringe 300 is formed from a second polymeric material. In the illustrative embodiment, the second polymeric material is more flexible than the first polymeric material. More specifically, in the illustrative embodiment, the first polymeric material forming the syringe body 330 comprises a food-grade polypropylene homopolymer (PPH), and the second polymeric material forming the cover member 320 comprises Food & Drug Administration (FDA)-approved, food-grade, virgin silicone. As such, in the illustrative embodiment, the first polymeric material forming the syringe body 330 and the second polymeric material forming the cover member 320 do not comprise latex, bisphenol A (BPA), or polyvinyl chloride (PVC) (i.e., the materials are latex-free, BPA-free, and PVC-free).

In the illustrative embodiment, the cover member 320, the syringe body 330, and the plunger assembly 340 are capable of being dissembled from one another for easy cleaning of the nasal irrigation syringe 300. As shown in the exploded view of FIG. 37, the cover member 320 and the syringe body 330 are able to be separated from one another so that each of the components 320, 330 are able to be separately cleaned with soap and water. Then, after the components 320, 330, 340 are cleaned, the cover member 320, the syringe body 330, and the plunger assembly 340 are able to be easily reassembled with one another.

Now, with reference to FIGS. 30-32, the manner in which the nasal irrigation syringe 300 of the illustrative embodiment is used will be described. Initially, a portion of the hemispherical protruding portion 322 of the cover member 320 is inserted into the nostril of a person (e.g., the nostril of a baby). After which, a user (e.g., a parent of the baby) expels the liquid solution (e.g., saline solution) from the fluid reservoir 338 of the syringe body 330 by pressing inwardly on the plunger handle portion 346 of the plunger assembly 340 to provide nasal irrigation for the person.

While the nasal irrigation syringe 300 is described for providing nasal irrigation for the person in the illustrative embodiment, it is to be understood that the syringe 300 may be used for many different applications requiring the dispensing of a liquid solution. Also, the innovative seal created by the compliant outer wall 323 disposed over the semi-rigid tip wall 333 of the tip member of the syringe body 330 may be used in a variety of different applications with many different dispensing devices.

It is readily apparent that the illustrative nasal irrigation syringe 300 offers numerous advantages. First, the nasal irrigation syringe 300 is easy to disassemble and clean. Secondly, the nasal irrigation syringe 300 includes primary and secondary stop portions 322, 326 for preventing the syringe 300 from being over-inserted into the nostril of the person. Finally, the irrigation syringe 300 forms an effective seal with the nostril of the person.

Advantageously, the aforedescribed nasal irrigation syringe 300 has three (3) detachable components 320, 330, 340 that can be disassembled and reassembled for effective cleaning. The nasal irrigation syringe 300 comprises a cover member 320 that acts as a barrier between the syringe tip member and the baby's nostril. This allows the device 300 to be comfortable for baby and allows parents to approach without fear of hurting the baby. Moreover, cover member 320 of the nasal irrigation syringe 300 also acts as a stabilizer for the parent using the device 300. The design of the cover member 320 provides an additional layer of safety for the baby when the device 300 is being used. In the illustrative embodiment, the application method is designed for the nasal irrigation syringe 300 to expel a saline solution (0.09% NaCl) into the baby's nostril. The cover member 320 using a silicone material creates a seal with baby's nostril, causing a rush of fluid to be rushed into the baby's nose and opening up the breathing pathways. This method will be easy to use, comfortable and smooth—flowing saline nasal irrigation for the baby.

Any of the features or attributes of the above described embodiments and variations can be used in combination with any of the other features and attributes of the above described embodiments and variations as desired.

Although the nasal device 10, 100, 200, 300 has been shown and described with respect to a certain embodiment or embodiments, it is apparent that the aforedescribed nasal device 10, 100, 200, 300 can be embodied in many different forms and that many other modifications and variations are possible without departing from the spirit and scope of the claimed invention.

Moreover, while exemplary embodiments have been described herein, one of ordinary skill in the art will readily appreciate that the exemplary embodiments set forth above are merely illustrative in nature and should not be construed as to limit the claims in any manner. Rather, the scope of the invention is defined only by the appended claims and their equivalents, and not, by the preceding description.

The invention claimed is:

1. A nasal device, comprising:
a tip member having a first end and a second end, the second end of the tip member being oppositely disposed relative to the first end, the tip member including a first wall disposed at the first end thereof, the first wall defining a first aperture configured to be in fluid communication with a nostril of a person;
a cover member disposed over a portion of the tip member, the cover member including a second wall overlapping at least a portion of the first wall of the tip member, the second wall defining a second aperture configured to be in fluid communication with the nostril of the person and the first aperture of the tip member, the cover member configured to provide a seal with the nostril of the person; and
a vacuum source or a fluid container coupled to the tip member, the vacuum source configured to create a vacuum force through the first and second apertures so as to remove mucus from the nostril of the person, or the fluid container configured to store a liquid solution that is to be expelled through the first and second apertures and into the nostril of the person.

2. The nasal device according to claim 1, wherein the tip member further comprises a tapered distal end portion at the first end thereof, the tapered distal end portion of the tip member including the first wall and being disposed inside the cover member.

3. The nasal device according to claim 2, wherein the tip member further comprises a stem portion connected to the tapered distal end portion.

4. The nasal device according to claim 3, wherein the stem portion of the tip member further comprises an annular protrusion disposed thereon, and the cover member further comprises a central passageway with an annular groove disposed in the central passageway, the annular protrusion on the stem portion of the tip member matingly engaging with the annular groove in the central passageway of the cover member so as to securely attach the cover member to the tip member.

5. The nasal device according to claim 3, wherein the tip member further comprises an attachment collar portion at the second end thereof, the stem portion being connected between the tapered distal end portion of the tip member and the attachment collar portion of the tip member, and the attachment collar portion attaching the tip member to the vacuum source or the fluid container.

6. The nasal device according to claim 5, wherein the nasal device comprises the vacuum source, the vacuum source being in a form of an aspirator bulb member, the aspirator bulb member being attached to the attachment collar portion of the tip member, the aspirator bulb member configured to create the vacuum force for removing mucus from the nostril of the person and through the first and second apertures, the vacuum force being created as a result of the aspirator bulb member being initially compressed by a user, and then the user gradually releasing the compression of the aspirator bulb member.

7. The nasal device according to claim 6, wherein the attachment collar portion of the tip member comprises one or more annular protrusions disposed thereon, the attachment collar portion of the tip member is received within an opening of the aspirator bulb member so as to attach the tip member to the aspirator bulb member, and the opening of the aspirator bulb member is circumscribed by a bounding wall with one or more annular grooves formed therein, the one or more annular protrusions on the attachment collar portion of the tip member matingly engaging with the one or more annular grooves in the bounding wall of the aspirator bulb member so as to securely attach the tip member to the aspirator bulb member.

8. The nasal device according to claim 5, wherein the nasal device comprises the vacuum source, the vacuum source being in a form of a mouth suction aspirator, and the mouth suction aspirator comprises:
a connector member that is attached to the attachment collar portion of the tip member;
a filter member disposed in the connector member; and
a mouthpiece connected to the connector member by means of a flexible tube, the mouth suction aspirator configured to create the vacuum force for removing mucus from the nostril of the person and through the first and second apertures when a user places the mouthpiece into his or her mouth and draws air through a distal end of the mouthpiece, the filter member configured to trap mucus from the nostril of the person so as to prevent the mucus, viruses, and/or bacteria from entering the mouth of the user.

9. The nasal device according to claim 8, wherein the filter member of the mouth suction aspirator comprises a serpentine air path for trapping the mucus from the nostril of the person inside the filter member.

10. The nasal device according to claim 5, wherein the nasal device comprises the fluid container, the fluid container being in a form of a nasal irrigation bottle, the attachment collar portion of the tip member being part of a bottle cap member, the nasal irrigation bottle configured to be attached to the bottle cap member, and the nasal irrigation bottle configured to hold the liquid solution for being expelled through the first and second apertures and into the nostril of the person.

11. The nasal device according to claim 10, further comprising a straw member extending from the tip member to a bottom of the nasal irrigation bottle so as to extract the liquid solution from the bottom of the nasal irrigation bottle.

12. The nasal device according to claim 2, wherein the nasal device comprises the fluid container, the fluid container being in a form of a nasal irrigation syringe, and the nasal irrigation syringe comprises:
a syringe body, the syringe body comprising a fluid reservoir for holding the liquid solution, and the tip member forming a part of the syringe body; and
a plunger assembly slidably received within the syringe body, the plunger assembly configured to be slidably displaced in a longitudinal direction of the syringe body, and the plunger assembly configured to discharge the liquid solution from the nasal irrigation syringe and into the nostril of the person when a force is applied to an end of the plunger assembly by a user.

13. The nasal device according to claim 12, wherein the plunger assembly comprises a plunger head portion, a plunger shaft having a cross-sectional profile, and a plunger handle portion, and the plunger handle portion of the plunger assembly is configured to receive the force applied by the user to discharge the liquid solution from the nasal irrigation syringe.

14. The nasal device according to claim 1, wherein the cover member further comprises a hemispherical protruding portion that includes the second wall, the hemispherical protruding portion operating as a primary stop portion sized to limit the depth that the second wall is able to be inserted into the nostril of the person, the hemispherical protruding portion further operating as a barrier between the tip member and the nostril of the person so as to enhance comfort for the person and prevent the tip member from directly contacting the nostril of the person, and a portion of the hemispherical protruding portion of the cover member configured to provide the seal with the nostril of the person.

15. The nasal device according to claim 14, wherein the cover member further comprises a cover body portion connected to the hemispherical protruding portion, the cover body portion operating as a stabilizer when the hemispherical protruding portion is being inserted into the nostril of the person, and the cover body portion further operating as a secondary stop portion sized to prevent over-insertion of the nasal device into the nostril of the person.

16. The nasal device according to claim 15, wherein the cover body portion is in a shape of an animal head.

17. The nasal device according to claim 16, wherein the animal head forming the cover body portion is a bear head.

18. The nasal device according to claim 1, wherein the tip member is formed from a first polymeric material, and the cover member is formed from a second polymeric material, the second polymeric material being more flexible than the first polymeric material.

19. The nasal device according to claim 18, wherein the first polymeric material forming the tip member comprises polypropylene, and the second polymeric material forming the cover member comprises silicone.

20. The nasal device according to claim 1, wherein the tip member, the cover member, and the vacuum source or the fluid container are capable of being dissembled from one another for easy cleaning of the nasal device.
